(19) United States
(12) Patent Application Publication
(10) Pub. No.: US 2024/0307009 A1
(43) Pub. Date: Sep. 19, 2024

(54) PREDICTION OF MECHANICAL FAILURE

(71) Applicant: Siemens Medical Solutions USA, Inc., Malvern, PA (US)

(72) Inventor: Martin Judenhofer, Knoxville, TN (US)

(21) Appl. No.: 18/184,958

(22) Filed: Mar. 16, 2023

(51) Int. Cl. A61B 6/03; A61B 6/00

(52) U.S. Cl. CPC A61B 6/035; A61B 6/482

(57) ABSTRACT

A framework for predicting mechanical failure. The framework may acquire vibration data from the at least one vibration sensor in a medical device. The vibration data may be pre-processed to generated pre-processed data. An onset of failure of the medical device may then be predicted based on the pre-processed data.

### TECHNICAL FIELD

The present disclosure generally relates to medical devices, and more particularly, to predicting mechanical failure in medical devices.

### BACKGROUND

The field of medical imaging has seen significant advances since the time X-Rays were first used to determine anatomical abnormalities. Medical imaging hardware has progressed from modern machines, such as Magnetic Resonance imaging (MRI) scanners, Computed Tomographic (CT) scanners and Positron Emission Tomographic (PET) scanners, to multimodality imaging systems such as PET-CT and PET-MRI systems.

Such medical devices typically contain mechanical components that are constantly subject to stress during operation. For example, MRI scanners use powerful magnetic field gradients to generate images of internal human organs. MRI scanners employ fast switching of currents in the gradient coils to enhance the imaging quality and shorten scanning time. However, such rapid switching of gradients produces forces that cause mechanical micro-motion in the gradient coils and their supports. As another example, CT scanners create images using a series of X-rays generated by an X-ray tube in a gantry that is rapidly rotated around the examined patient. The use of slip rings in a gantry allows continuous complete circular movements of the internal elements without the internal circuits and cables becoming entangled. However, the rotating gantry causes vibrations that expose the gantry and support components to stress over time.

Vibration-induced stress can result in mechanical failure in the components of a medical device. Failure to detect the imminent onset or existence of such stress-related failure conditions and perform regular maintenance can lead to extended downtime and high repair expenses.

### SUMMARY

Described herein is a framework for predicting mechanical failure. The framework may acquire vibration data from the at least one vibration sensor in a medical device. The vibration data may be pre-processed to generated pre-processed data. An onset of failure of the medical device may then be predicted based on the pre-processed data.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary system;

FIG. 2 shows an exemplary method of predicting mechanical failure;

FIG. 3 shows an exemplary waveform in the time-domain in a simulated

normal scenario;

FIG. 4 shows an exemplary spectrum in the frequency-domain in a simulated normal scenario;

FIG. 5 shows an exemplary waveform in the time-domain in a simulated imminent failure scenario; and

FIG. 6 shows exemplary spectrum in the frequency-domain in a simulated imminent failure scenario.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed; on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

Unless stated otherwise as apparent from the following discussion, it will be appreciated that terms such as “segmenting,” “generating,” “registering,” “determining,” “aligning,” “positioning,” “pre-processing”, “processing,” “computing,” “selecting,” “estimating,” “detecting,” “tracking” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and interface to a variety of operating systems. In addition, implementations of the present framework are not described with reference to any particular programming language. It will be appreciated that a variety of programming languages may be used.

As used herein, the term “image” refers to multi-dimensional data composed of discrete image elements (e.g., pixels for 2D images and voxels for 3D images). The image may be, for example, a medical image of a subject collected by computer tomography, magnetic resonance imaging, ultrasound, or any other medical imaging system known to one skilled in the art. The image may also be provided from non-medical contexts, such as, for example, remote sensing systems, electron microscopy, etc. Although an image can be thought of as a function from R³ to R, or a mapping to R³, the present methods are not limited to such image and can be applied to images of any dimension, e.g., a 2D picture or a 3D volume. For a 2-or 3-dimensional image, the domain of the image is typically a 2-or 3-dimensional rectangular array, wherein each pixel or voxel can be addressed with reference to a set of 2 or 3 mutually orthogonal axes. The terms “digital” and “digitized” as used herein will refer to images or volumes, as appropriate, in a digital or digitized format acquired via a digital acquisition system or via conversion from an analog image.

One aspect of the present framework provides active measurement of vibration data acquired by a vibration sensor (e.g., microelectromechanical (MEMS)-based accelerometer) located in a medical device or one or more components thereof. In some implementations, the vibration data is used to perform a first order detection of a static change (e.g., change in orientation or gravitational force direction), which can be due to a failure or loosening in mounting. Alternatively, or in combination thereof, the vibration data is used to perform a higher order detection of a dynamic change (e.g., change in frequency or amplitude) in response to mechanical stimulation of the medical device. The time and/or frequency patterns (or profiles) based on such vibration data may be analyzed to determine whether the underlying mechanical system has undergone a change in stiffness (e.g., weakening or loosening of a component).

Embedding or adding such vibration sensor into the medical device can enable early detection of mechanical failure that is realized or starting to develop, such as when a component is starting to, for example, move, loosen, crack and change in stiffness, while not being at the point of full failure. This can allow for more accurate prediction of needed maintenance and extend component lifetime, since each component may undergo site-specific stresses that may not be properly accounted for in conventional prediction models, or when models are using a worst-case estimate to allow for a large enough safety margin. As a result of more accurate failure prediction, cost of the medical device over its lifetime is reduced. These and other features and advantages will be described in more detail herein.

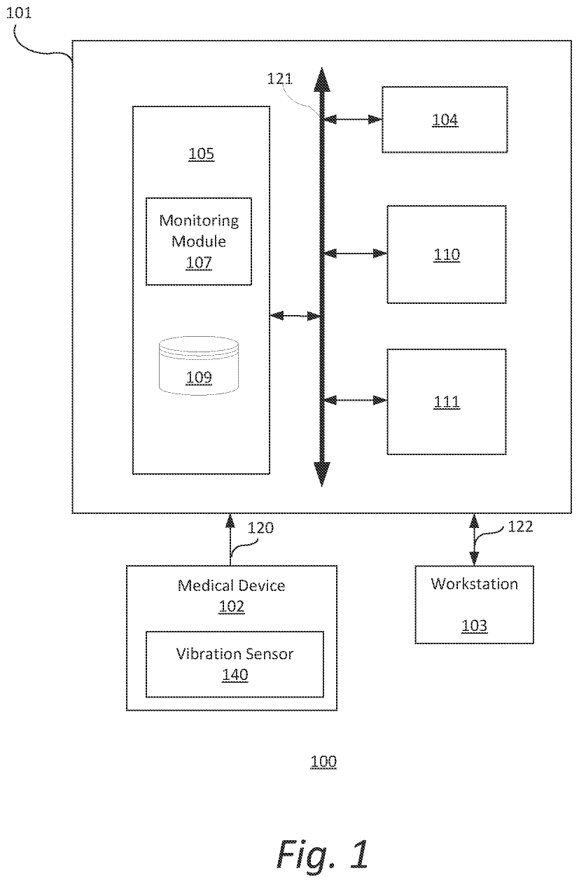

FIG. 1 is a block diagram illustrating an exemplary system 100. System 100 includes a computer system 101 for implementing the framework as described herein. In some implementations, computer system 101 operates as a standalone device. In other implementations, computer system 101 may be connected (e.g., using a network) to other machines, such as medical device 102 and workstation 103. In a networked deployment, computer system 101 may operate in the capacity of a server (e.g., thin-client server), a cloud computing platform, a client user machine in server-client user network environment, or as a peer machine in a peer-to-peer (or distributed) network environment.

In some implementations, computer system 101 comprises a processor or central processing unit (CPU) 104 coupled to one or more non-transitory computer-readable media 105 (e.g., computer storage or memory device), a display device 110 (e.g., monitor) and various input devices 111 (e.g., mouse or keyboard) via an input-output interface 121. Computer system 101 may further include support circuits such as a cache, a power supply, clock circuits and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in non-transitory computer-readable media 105. In particular, the present techniques may be implemented by a monitoring module 107.

Non-transitory computer-readable media 105 may include random access memory (RAM), read-only memory (ROM), magnetic floppy disk, flash memory, and other types of memories, or a combination thereof. The computer-readable program code is executed by CPU 104 to process data retrieved from, for example, medical device 102, workstation 103 and database 109. As such, computer system 101 is a general-purpose computer system that becomes a specific-purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein.

The same or different computer-readable media 105 may be used for storing a database (or dataset) 109. Such data may also be stored in external storage or other memories. The external storage may be implemented using a database management system (DBMS) managed by the CPU 104 and residing on a memory, such as a hard disk, RAM, or removable media. The external storage may be implemented on one or more additional computer systems. For example, the external storage may include a data warehouse system residing on a separate computer system, a cloud platform or system that provides off-site or remote storage, a picture archiving and communication system (PACS), or any other hospital, medical institution, medical office, testing facility, pharmacy or other medical patient record storage system.

Workstation 103 may include a tablet, mobile device, laptop or computer intended to be used by the medical technician. Workstation 103 may include a graphical user interface to receive user input via an input device (e.g., keyboard, mouse, touch screen, voice or video recognition interface) to input medical data 122. Workstation 103 can be operated in conjunction with the entire system 100. For example, workstation 103 may communicate directly or indirectly with the medical device 102.

Medical device 102 is any device that is used for medical purposes. In some implementations, medical device 102 is a radiological medical scanner that acquires medical image data 120 associated with patients. Medical device 102 may acquire medical image data 120 using techniques such as, but not limited to, high-resolution computed tomography (HRCT), magnetic resonance (MR) imaging, computed tomography (CT), helical CT, X-ray, angiography, positron emission tomography (PET), fluoroscopy, single photon emission computed tomography (SPECT), or a combination thereof.

Medical device 102 has one or more components that are subject to mechanical stimulation (or vibration) during operation of the medical device 102. In some implementations, at least one vibration sensor 140 is removably attached to at least one of the components of medical device 102 subject to mechanical stimulation. Alternatively, the at least one vibration sensor 140 is embedded in at least one of the components of medical device 102 subject to mechanical stimulation. The at least one vibration sensor 140 acquires vibration data 120 of the medical device 102 or component thereof. Vibration sensor 140 may include, for example, a 3D MEMS accelerometer that can record vibration (or change in motion or force) with sampling speed in the kHz range (e.g., about 1-5 kHz). Other types of vibration sensors 140, such as piezo-based sensors (e.g., piezoelectric sensor, piezoresistive sensor), capacitive sensors or strain gauge type sensors, are also useful.

The at least one vibration sensor 140 may be located in, for example, a medical scanner component, such as a detector (e.g., PET, CT detector), an X-Ray source, gradient coil, a body coil or a gantry (e.g., in a CT scanner). As another example, vibration sensor 140 may be located on a printed circuit board (PCB) inside a combined PET-MRI gantry, wherein the vibration sensor 140 operates in a 3T magnetic field without detriment. Multiple vibration sensors 140 may be located on different components of the medical device 102.

The at least one vibration sensor 140 may be loosely bonded, affixed or cast into the medical device component, so that it can pick up the vibration pattern. The vibration data acquired by the at least one sensor 140 may be used to perform data analysis (e.g., time time-frequency-analysis) under mechanical stimulation (e.g., by applying or switching a gradient, while rotating the CT gantry, or by some other mechanical stimulus). Monitoring the vibration patterns allows the monitoring module 107 to track changes and determine a mechanical failure or the onset of an imminent failure.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

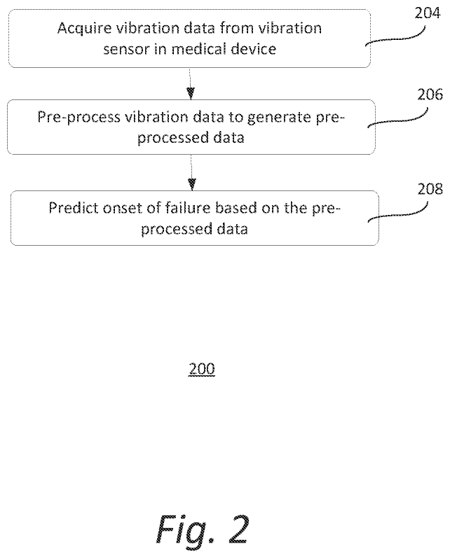

FIG. 2 shows an exemplary method 200 of predicting mechanical failure. It should be understood that the steps of the method 200 may be performed in the order shown or a different order. Additional, different, or fewer steps may also be provided. Further, the method 200 may be implemented with the system 100 of FIG. 1, a different system, or a combination thereof.

At 204, monitoring module 107 acquires vibration data 120 from at least one vibration sensor 140 in medical device 102. Vibration data 120 is indicative of vibration of the medical device 102 or a component thereof. Such vibration data 120 may include, for instance, orientation data, acceleration data, velocity data or position data. Vibration data 120 may be acquired over time to detect small changes. Vibration data 120 may be acquired along three orthogonal axes (e.g., X-axis, Y-axis, Z-axis). Multiple vibration sensors 140 may be positioned on different components of the medical device 102 to acquire different sets of vibration data 120 for monitoring the health of the different components. Vibration data 120 may be transmitted to monitoring module 107 via, for example, a universal serial bus (USB) or an Inter-Integrated Circuit (I2C) data bus. Other types of data transfer, such as Serial Peripheral Interface (SPI), Wi-Fi or Bluetooth, are also useful.

In some implementations, monitoring module 107 initiates mechanical stimulation of medical device 102 prior to acquiring vibration data 120. Mechanical stimulation of the medical device 102 causes vibrations that are propagated and detected by at least one vibration sensor 140 in medical device 102. Mechanical stimulation may be initiated by operating a medical scanner component. For example, mechanical stimulation may be initiated by applying or switching a gradient (e.g., starting an MRI sequence, spin echo sequence, dedicated service sequence) in the medical device 102 (or scanner). As another example, mechanical stimulation may be initiated by starting rotation of a CT scanner gantry.

At 206, monitoring module 107 pre-processes the vibration data 120 to generate pre-processed data. The vibration data 120 may be transformed from the time domain to the frequency domain. This may be performed by, for example, performing the Fast Fourier Transform (FFT) on the vibration data 120 to generate a frequency spectrum. In some implementations, pre-processing operations, such as normalization, amplification, smoothing and/or filtering operations to remove noise, may be performed on the vibration data 120. For example, filtering may be performed to retain only the strong components of the vibration data 120. Other types of pre-processing operations are also useful.

At 208, monitoring module 107 predicts the onset of failure of the medical device based on the pre-processed data. In some implementations, the prediction of the onset of failure is performed by performing a first order detection of a static change in the pre-processed data without initiating any prior mechanical stimulation of the medical device or component thereof. The pre-processed data may indicate a change in orientation or gravitational force direction, which can be due to a failure or loosening in mounting in the medical device 102.

Alternatively, or in combination thereof, the pre-processed data is used to perform a higher order detection of a dynamic change (e.g., change in frequency or amplitude) in response to mechanical stimulation of the medical device or component thereof. The time and/or frequency patterns (or profiles) based on such pre-processed data may be analyzed to determine whether the underlying mechanical system has undergone a change in stiffness (e.g., weakening or loosening of a component).

The pre-processed data may be represented as a waveform in the time domain, a spectrum in the frequency domain, or a combination thereof. Monitoring module 107 may monitor the time waveform of the pre-processed data in the time domain for any changes that are generated in response to the mechanical stimulus. For instance, when the stiffness of the medical device 102 (or component thereof) drops due to imminent failure (e.g., weakening or loosening of a component), the maximum amplitude of the time waveform may be lower with more peaks occurring over a longer period of time.

Monitoring module 107 may also monitor the frequency spectrum of the pre-processed data in the frequency domain for any changes generated in response to the mechanical stimulus. For instance, when the stiffness of the medical device 102 (or component thereof) drops before the onset of failure, the maximum amplitude of the frequency spectrum may be higher with fewer peaks occurring over a shorter bandwidth of frequencies. Other types of data analysis, such as phase measurement, order analysis, power spectral density analysis, envelope analysis, or a combination thereof, are also useful.

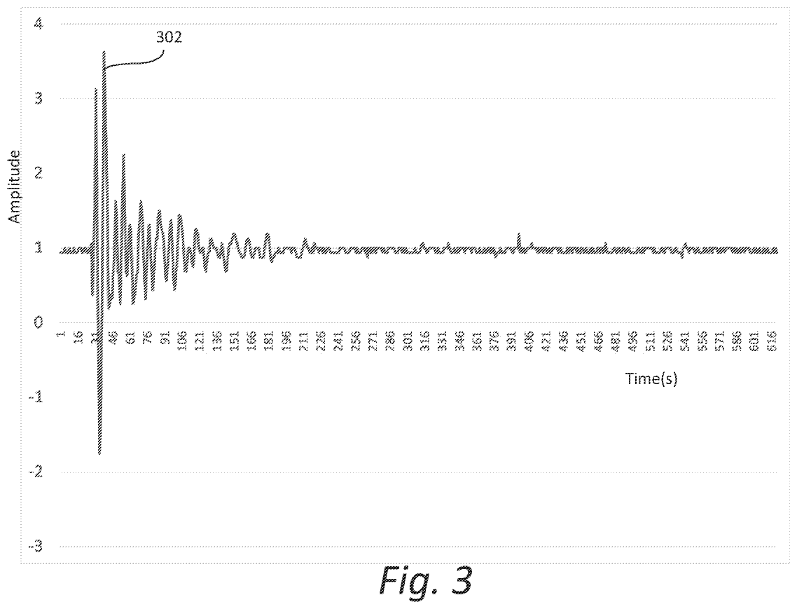

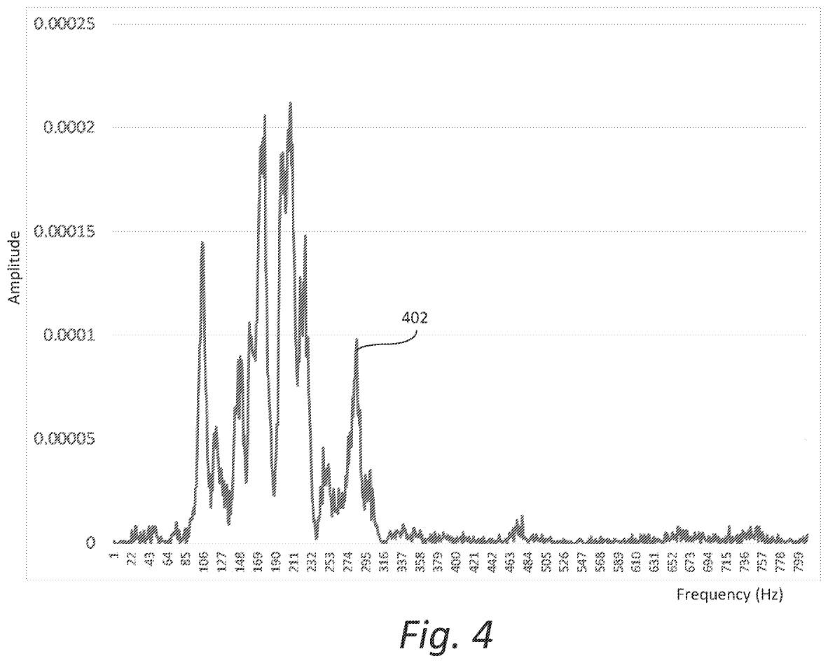

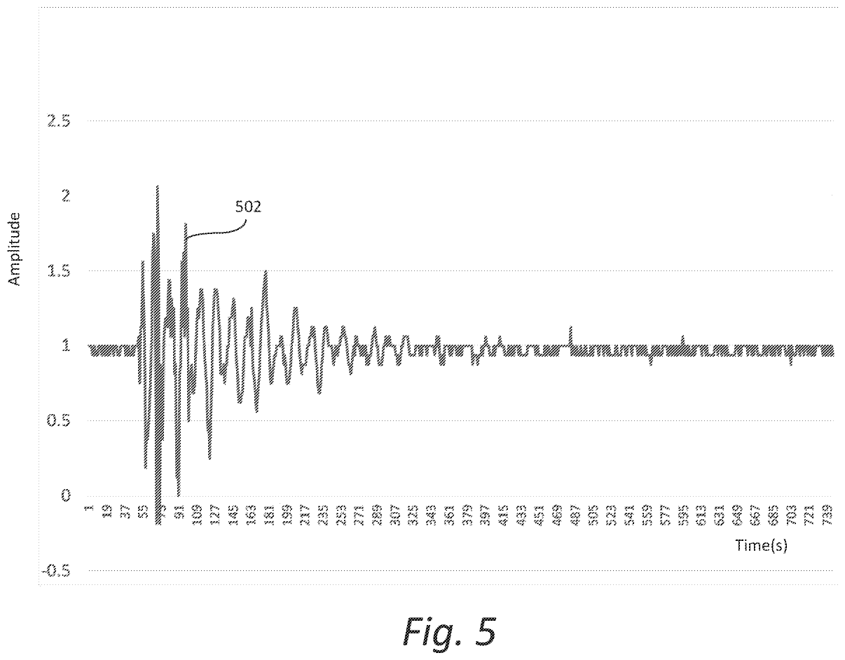

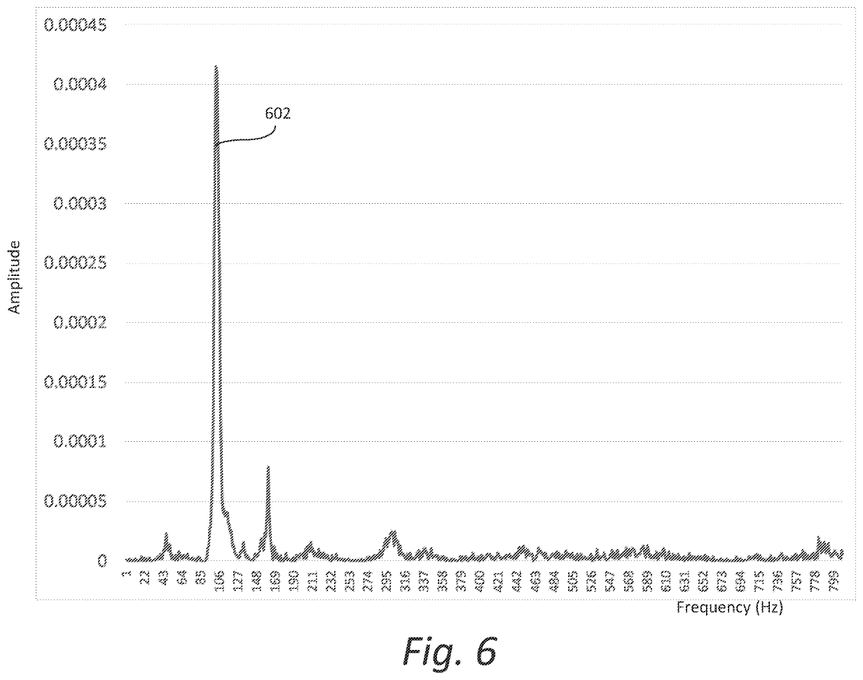

FIG. 3 shows exemplary waveform 302 in the time-domain, while FIG. 4 shows an exemplary spectrum 402 in the frequency-domain in a simulated normal (i.e., no fault) scenario. Waveform 302 and spectrum 402 were generated in response to a mechanical stimulus of a simulated medical device when there is no imminent failure. FIG. 5 shows an exemplary waveform 502 in the time-domain, while FIG. 6 shows an exemplary spectrum 602 in the frequency-domain in a simulated imminent failure scenario. Waveform 502 and spectrum 602 were generated in response to a mechanical stimulus of a simulated medical device before the onset of failure. The mechanical stimulus in these scenarios is a knock on a printed circuit board (PCB) with an attached vibration sensor, which simulates the switching of a gradient during an MRI sequence in a medical device.

In the imminent failure scenario illustrated by FIGS. 5 and 6, the simulated medical device arrangement has a lower stiffness than during the normal scenario. In the time domain, the mechanical stimulus produced a waveform 502 with lower amplitudes than the waveform 302 in the normal scenario. In the frequency domain, the mechanical stimulus produced a spectrum 602 with a higher peak amplitude and fewer peaks in a narrower bandwidth of vibration frequencies, while the spectrum 402 in the normal scenario has more peaks within a broader bandwidth of vibration frequencies.

Accordingly, by monitoring changes in vibration amplitude and the shifts in frequencies, imminent failure may be predicted. Changes in amplitude or frequency of vibration, assuming the same mechanical stimulus, indicate a change to the underlying stiffness of the medical device 102, since the frequency and amplitude are a function of the system stiffness. Such changes can indicate mechanical damage and/or the onset of mechanical failure. Observations of small changes over time before the onset of failure may be good predictors of imminent failure.

Using different mechanical stimuli (e.g., pulse excitation, vibration excitation), different types of failures (e.g., loosening of particular fastening points, breakage and tear of components) and/or different locations of the vibration sensor may also produce different responses. Machine learning or artificial intelligence tools (e.g., convolutional neural networks) may also be used to predict the onset of failure based on the pre-processed data using feature extraction and classification techniques. Preventive maintenance intervals may then be estimated based on the predicted onset of failure. For example, when imminent failure is detected, an alert may be generated and maintenance personnel may be scheduled based on availability to perform preventive maintenance tasks.

Steps 204 through 208 may be repeated as often as needed to detect any imminent onset or existence of failure. The steps 204-208 may be repeated as often as feasible (e.g., once a day) or routinely whenever medical device 102 is operated to perform medical imaging of a patient.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. A system for mechanical failure prediction, comprising:
a non-transitory memory device for storing computer-readable program code; and
a processor in communication with the non-transitory memory device, the processor being operative with the computer-readable program code to perform operations including
acquiring vibration data from at least one vibration sensor in a medical device,
pre-processing the vibration data to generate pre-processed data, and
predicting an onset of failure of the medical device based on the pre- processed data.

2. The system of claim 1 wherein the medical device comprises a radiological medical scanner.

3. The system of claim 1 wherein the at least one vibration sensor is located in a detector, an X-ray source, a gradient coil, a body coil or a gantry of the medical device.

4. The system of claim 1 wherein the at least one vibration sensor is located on a printed circuit board inside a gantry of the medical device.

5. The system of claim 1 wherein the at least one vibration sensor is removably attached to at least one component of the medical device.

6. The system of claim 1 wherein the at least one vibration sensor is embedded in at least one component of the medical device.

7. The system of claim 1 wherein the at least one vibration sensor comprises a microelectromechanical-based accelerometer.

8. The system of claim 1 wherein the processor is further operative with the computer-readable program code to
initiate mechanical stimulation of the medical device prior to acquiring the vibration data from the at least one vibration sensor.

9. A method of predicting mechanical failure, comprising:
acquiring vibration data from at least one vibration sensor in a medical device;
pre-processing the vibration data to generate pre-processed data; and
predicting an onset of failure of the medical device based on the pre-processed data.

10. The method of claim 9 wherein predicting the onset of failure comprises detecting a static change in the pre-processed data.

11. The method of claim 10 wherein detecting the static change in the vibration data comprises detecting a change in orientation.

12. The method of claim 9 wherein predicting the onset of failure comprises detecting a dynamic change in the pre-processed data in response to a mechanical stimulation of the medical device.

13. The method of claim 12 further comprising:
initiating the mechanical stimulation of the medical device prior to acquiring the vibration data from the at least one vibration sensor.

14. The method of claim 13 wherein initiating the mechanical stimulation of the medical device comprises operating a medical scanner component.

15. The method of claim 14 wherein operating the medical scanner component comprises switching a gradient in the medical scanner component.

16. The method of claim 14 wherein operating the medical scanner component comprises starting rotation of a computed tomographic (CT) scanner gantry.

17. The method of claim 9 wherein acquiring the vibration data from the at least one vibration sensor comprises acquiring orientation data, acceleration data, velocity data, position data or a combination thereof.

18. The method of claim 9 wherein predicting the onset of failure of the medical device based on the pre-processed data comprises monitoring changes in orientation, amplitude or frequency indicated by the pre-processed data.

19. The method of claim 9 wherein predicting the onset of failure of the medical device based on the pre-processed data comprises monitoring a time waveform or frequency spectrum of the pre-processed data.

20. One or more non-transitory computer readable media embodying a program of instructions executable by machine to perform operations for predicting mechanical failure, the operations comprising:
acquiring vibration data from at least one vibration sensor in a medical device;
pre-processing the vibration data to generate pre-processed data; and
predicting an onset of failure of the medical device based on the pre-processed data.
